(19) United States
(12) Patent Application Publication
(10) Pub. No.: US 2022/0401610 A1
(43) Pub. Date: Dec. 22, 2022

(54) OZONE SYSTEM FOR THE INACTIVATION OF BACTERIA AND VIRUSES

(71) Applicants: Daniel S. Kissel, Brookfield, IL (US); Timothy Gognat, Lakewood, CO (US); Michael Gordon Scott, Colorado Springs, CO (US); Patrick Scher, Golden, CO (US); Christopher J. Hooker, Plainfield, IL (US); Mallory A. Havens, Homer Glen, IL (US); Gary R. Burleson, Raleigh, NC (US); Ryan Phillips, Yorkville, IL (US)

(72) Inventors: Daniel S. Kissel, Brookfield, IL (US); Timothy Gognat, Lakewood, CO (US); Michael Gordon Scott, Colorado Springs, CO (US); Patrick Scher, Golden, CO (US); Christopher J. Hooker, Plainfield, IL (US); Mallory A. Havens, Homer Glen, IL (US); Gary R. Burleson, Raleigh, NC (US); Ryan Phillips, Yorkville, IL (US)

(21) Appl. No.: 17/581,223

(22) Filed: Jan. 21, 2022

Related U.S. Application Data
(60) Provisional application No. 63/139,855, filed on Jan. 21, 2021.

(51) Int. Cl. A61L 9/12; C01B 13/11; A61L 2/20

(52) U.S. Cl. CPC A61L 9/122; C01B 13/11; A61L 2/202

(57) ABSTRACT

An ozone treatment system which is usable in enclosed or confined spaces for the inactivation of pathogens such as bacteria and viruses in these spaces.

### CROSS REFERENCE TO RELATED APPLICATIONS

This application priority from U.S. Provisional Patent Application 63/139,855, filed Jan. 21, 2021, the disclosure of which is incorporated herein by reference in its entirety.

### FIELD OF THE INVENTION

The invention relates to an ozone system which is usable in enclosed or confined spaces for the inactivation of pathogens such as bacteria and viruses in these spaces.

### BACKGROUND OF THE INVENTION

Ozone is a powerful oxidant that has many industrial and consumer applications related to oxidation. It has been shown to inactivate a large number of pathogens including bacteria, viruses and fungi. Ozone is formed from diatomic oxygen by ultraviolet light or electrical discharges. Commercially available ozone generators use oxygen from the air or an O₂ tank to create ozone. Ozone is an unstable triatomic molecule, O₃, that readily decomposes to oxygen naturally, with a half-life reported at ˜30 minutes in room temperature environments where air changes vary between 5 and 8 Air Changes per Hour (ACH). Because of its strong oxidizing potential, ozone can also cause damage to mucous and respiratory tissues in animals and humans. The EPA guidelines suggest humans not be exposed to an average concentration of 0.1 ppm for more than 8 hours.

FIG. 1 shows one known ozone generator developed for hotel room sterilization. However, this design requires an extended period of time for use, wherein sterilization and decomposition of ozone of a hotel room may take several hours. This generator sold under the name Viroforce 1000 contained eight corona discharge units, a circulation fan, and a catalytic converter to convert ozone back to oxygen after treatment. FIG. 1 shows the unit by itself and in a hotel room.

It is an object of the invention to provide an improved system for generating and distributing ozone that overcomes disadvantages associated with known ozone generators.

The invention relates to an inventive system and method of ozone generation and distribution, which incorporates an ozone generator to generate ozone in a confined treatment space for the inactivation or killing of pathogens such as bacteria and viruses. The ozone is maintained by the system for a time period suitable to inactivate the pathogens in the treatment space. The inventive system further includes an improved system for decomposing the ozone in the treatment space after the space has been treated for pathogens. The decomposition system includes a converter unit having a novel reactive catalyst that decomposes ozone more quickly to allow reuse of the treatment space after the ozone treatment is completed.

### BRIEF DESCRIPTION OF THE DRAWINGS

FIG. 1 illustrates a known ozone generation system for hotel rooms.

FIG. 2 is a diagrammatic view of a traditional generator system for ozone production.

FIG. 3 is a representative view of a SARS-CoV-2 virus structure representing one targeted pathogen for the present invention.

FIG. 4 is a table showing permissible OSHA/US EPA ozone limits and exposure times.

FIG. 5 is a graph of the ozone half-life for a control test and subsequent tests using a novel catalyst in a converter unit used in a treatment system according to the present invention.

FIG. 6 is a graph showing the ozone concentration vs. time for a remotely operated ozone treatment with an activated carbon catalyst.

FIG. 7A is a pictorial view of the treatment system in use in an airplane cabin.

FIG. 7B is schematic diagram of the inventive ozone generator incorporated into an airflow system.

FIG. 8A is a perspective view of an alternate ozone treatment generation system.

FIG. 8B is a perspective view of the alternate system with a cover removed.

FIG. 9 is a diagrammatic view of the treatment system and the testing locations in the airplane cabin.

Certain terminology will be used in the following description for convenience and reference only, and will not be limiting. For example, the words “upwardly”, “downwardly”, “rightwardly” and “leftwardly” will refer to directions in the drawings to which reference is made. The words “inwardly” and “outwardly” will refer to directions toward and away from, respectively, the geometric center of the arrangement and designated parts thereof. Said terminology will include the words specifically mentioned, derivatives thereof, and words of similar import.

### DETAILED DESCRIPTION

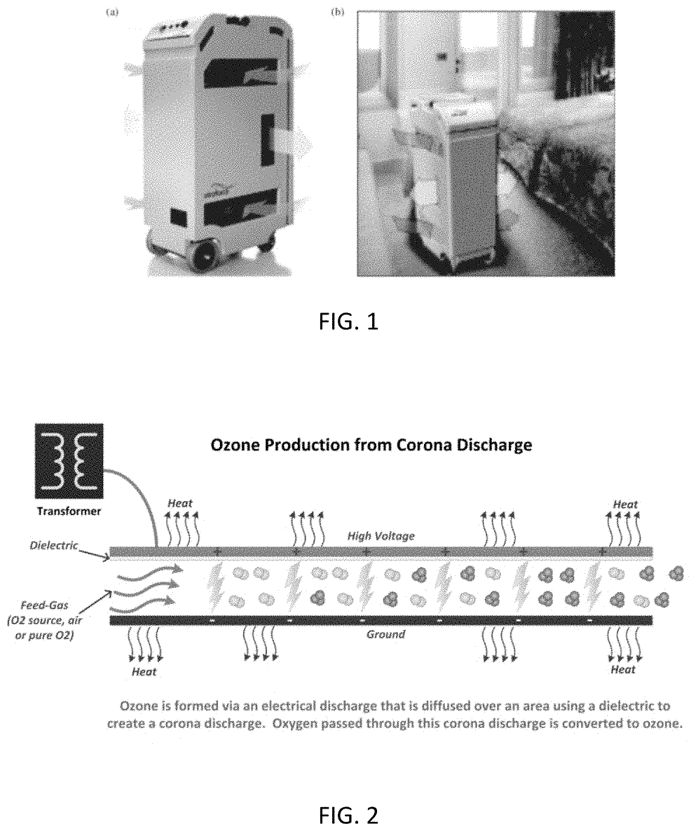

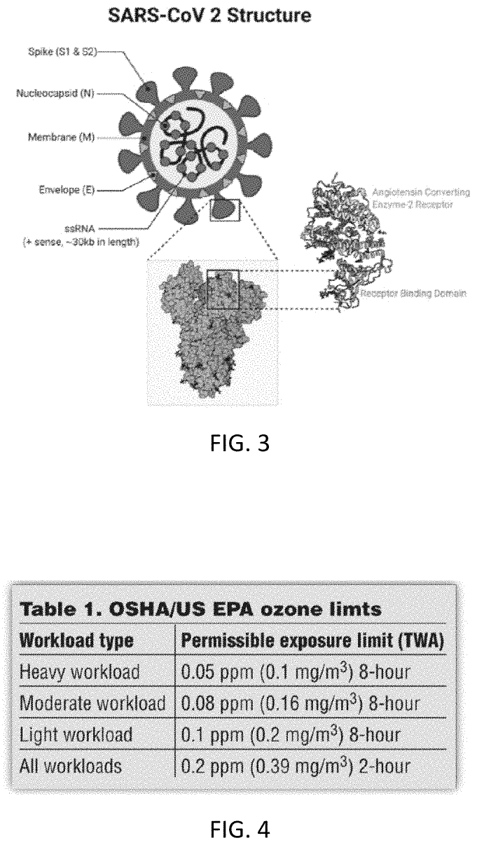

Referring to FIG. 1, the invention relates to an improved system and method for inactivating pathogens in enclosed spaces through the use of ozone. The inventive system is provided to generate a high enough concentration of ozone for suitable pathogen inactivation, equally disperse ozone within the applied space for a specified unit of time, and quickly decompose the ozone in the treated space using remote operation. FIG. 1 illustrates an ozone generation system wherein ozone is formed via an electrical discharge, preferably from a transformer or other electrical source. A feed gas, such as air, purified O₂ or some other source of O₂, is supplied to an ozone generation chamber through which the electrical discharge flows. The ozone is formed by the electrical discharge that is diffused over an area using a dielectric, wherein a corona discharge is created within the ozone generation chamber. During operation, the oxygen that passes through the corona discharge is converted to ozone.

As a triatomic molecule, ozone also functions as an antibacterial, virucide, and fungicide. Over time, the ozone decomposes to safe oxygen such that its effects are effective but temporary. Ozone is easy to generate from ambient air or from an O₂ feed gas.

As noted, ozone is effective in inactivating or killing various pathogens. These pathogens can include the following:
1)Viruses—Ozone destroys viruses by diffusing through the protein coat into the nucleic acid core, where it damages viral RNA. At higher concentrations, ozone destroys the virus' exterior protein shell so that DNA or RNA structures are affected.
2) Bacteria—Ozone interferes with bacterial cell metabolism, likely through inhibition of the enzymatic control system.
3) Fungi including Molds and Yeasts—It is believed that ozone destroys fungi by diffusing through the fungal wall and into the cytoplasm, disrupting the organelles that direct cell function.

FIG. 2 illustrates the SARS-CoV-2 Structure, which is one pathogen with which the present invention is effective. The ozone generated by the present invention is believed to inactivate this pathogen by oxidation of lipid membrane and sulfur atoms on RNA strands, diffusion into the nucleic acid core and deactivation of viral RNA, and inactivation of viral binding spikes.

The invention therefore relates in part to a treatment system for generating ozone to sanitize confined treatment spaces. In a preferred embodiment, the treatment system is configured as a remotely operated ozone generator having an ozone generator unit combined with an airflow system and converter unit for expediting the distribution and decomposition of the ozone generated. The treatment system is configured for the treatment of confined spaces, such as airplane cabins for sterilization of airplanes in between flights, or closed cabinets, which permits sterilization of PPE inside of the sealed cabinets or other types of spaces. The inventive system can sanitize these spaces and the materials or surfaces therein while avoiding the presence of people.

The treatment system is configured so that it can generate ozone at a specific concentration and hold this specific concentration within suitable tolerances for a specific time period that is suitable for appropriate pathogen inactivation. The treatment system includes an air handling unit that can rapidly move air to equally distribute ozone during ozone generation. Further, the treatment system may include a converter unit that may be formed as a catalytic converter to convert ozone back to oxygen. The converter unit preferably incorporates a novel catalyst developed as an inventive component of the treatment system to quickly decompose ozone back to oxygen and reduce ozone levels so as to restore the treatment spaces to a condition safe for people.

The novel catalyst expedites conversion of the ozone to oxygen so that the treatment system can operate within a smaller or reduced time frame in comparison to natural ozone decomposition to complete sanitation and conversion of ozone back to oxygen in under 30 minutes, which could not occur if relying on natural decomposition. FIG. 4 lists the ozone limits established by US government agencies.

In operation, effective viral inactivation is a function of ozone concentration and exposure time using the following formula:

[O₃]mg/m³×Time(min)=50

According to the present invention, the preferred target concentration is 5 mg/m³ or greater of ozone, which is held by the treatment system at this level for an appropriate minimum time based upon this formula.

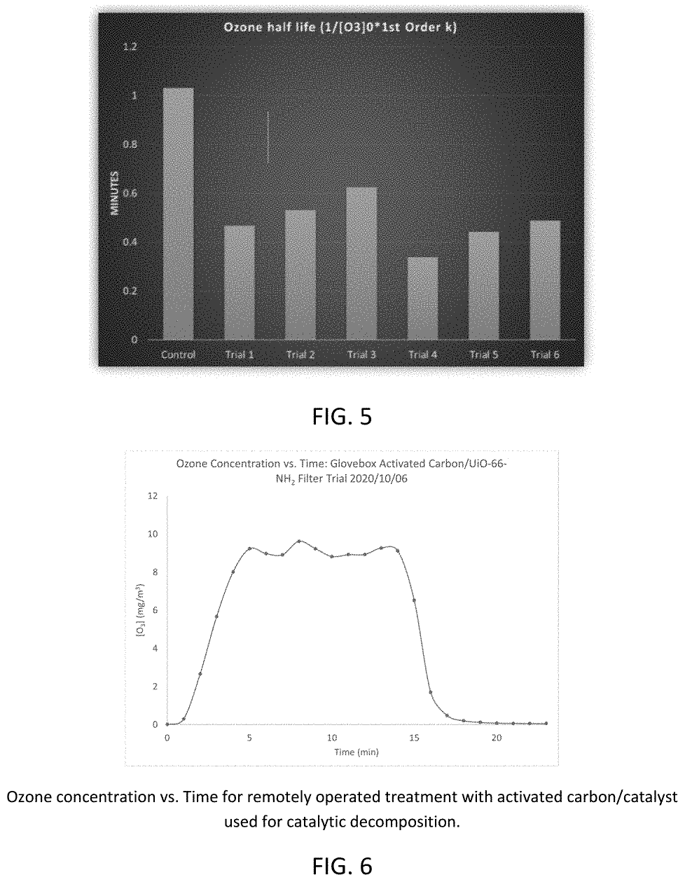

As an additional feature of the treatment system, the ozone converter unit includes a novel catalyst which efficiently converts ozone back to oxygen, which thereby expedites return of the treatment space back to a safe condition for humans to enter or access. The catalyst is a material specifically described as a Metal-Organic Framework (MOF) serving as a reactive catalyst in the converter unit. Preferably, the MOF is the substance, UiO-66-NHO₂. FIG. 5 illustrates 6 trials using this catalyst as part of the treatment system. As noted, the control half-life exceeds 1 minute, while operation of the treatment system with the reactive catalyst reduces the half-life to near or below 30 seconds. Further, doubling the catalyst mass increases performance by further reducing the half-life.

In some embodiments, the catalyst may also include an additional adsorbent media, such as activated carbon (C) or zeolites, blended with the novel catalyst. FIG. 6 is a graph showing ozone concentration versus time inside a sealed cabinet that displays generation and decomposition using a blend of activated carbon and UiO-66-NHO₂ to catalyze the decomposition of ozone back to oxygen. The generation, distribution and catalytic conversion was accomplished using the inventive system with remote operation. The total time for ozone generation, treatment and decomposition during this process was less than 20 minutes.

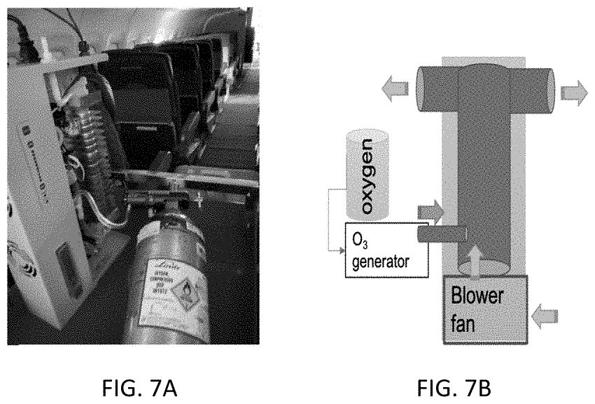

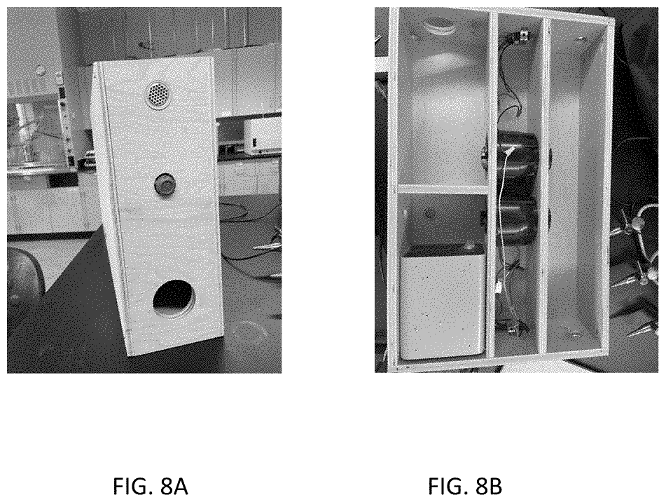

Further large scale testing was performed on an airplane cabin which served as the treatment space using Escherichia coli (E. coli) as a model pathogen. During testing of the treatment method and system, E. coli was swabbed onto growth media in petri dishes and placed throughout the treatment space. The trials were completed by treatment using 5.0 mg/m³ (2.5 ppm at room temperature) of ozone for 10 minutes. E. coli samples from the trials exposed to ozone in the treatment space were analyzed and compared to control samples not subjected to the treatment method to determine kill rates. The treatment unit utilized for trials completed on the interior of an American Airlines MD80 airplane is shown in FIG. 7A, which includes an ozone generator supplied with oxygen by a supply tank or other source. The treatment system also may include a distribution unit for distributing a flow of ozone into the space. The distribution unit may be a fan blower or other air handler that may generate an airflow through a pipe or passage wherein an ozone generator forms ozone within the passage, and the airflow distributes the ozone to the treatment space in accord with FIG. 7B. FIGS. 8A and 8B shows an alternate treatment system created for smaller aircraft spaces using an ozone generator integrated into the airflow distribution unit.

In use, the ozone generator creates and pumps or blows the ozone to fill the treatment space, i.e., the airplane cabin. As noted, the passage may flow through a plate generator that is part of the ozone generator, wherein one or more plate generators may be provided. The oxygen source may in turn feed the generator, which may be pure oxygen, into the passage for conversion to ozone. The treatment system may also include a detector or sensor unit to monitor the concentration of ozone within the treatment space and automatically adjust the ozone generator and flow of ozone into the treatment space to maintain the ozone at the selected concentration. The system may also adjust the concentration higher or lower as desired.

As noted above, the treatment system may also include the converter unit with a catalyst in accord with the description provided herein. The ozone may first be held at a desired concentration for a selected time period during a sanitizing or deactivation cycle during which pathogens are inactivated by the ozone. Preferably, the ozone is maintained at about 2.5 to 3 ppm (5.0 to 6.0 mg/m³ at room temperature) for 10 minutes although other concentrations and treatment times may be selected. The catalytic converter unit may then be operated to convert the ozone back to oxygen to restore a safe concentration of ozone in the treatment space, which preferably is 0.1 ppm (0.2 mg/m³ at room temperature).

During testing, the inactivation cycle might take significantly longer, such as 45 minutes, when only the ozone generator is used to sanitize the airplane cabin and then the ozone is allowed to naturally covert back to oxygen without a catalyst. When the inventive treatment is operated with the converter unit and MOF catalyst, the entire cycle of sanitizing and converting ozone can be reduced to about 25 minutes or less due to the reduction in the ozone half-life time.

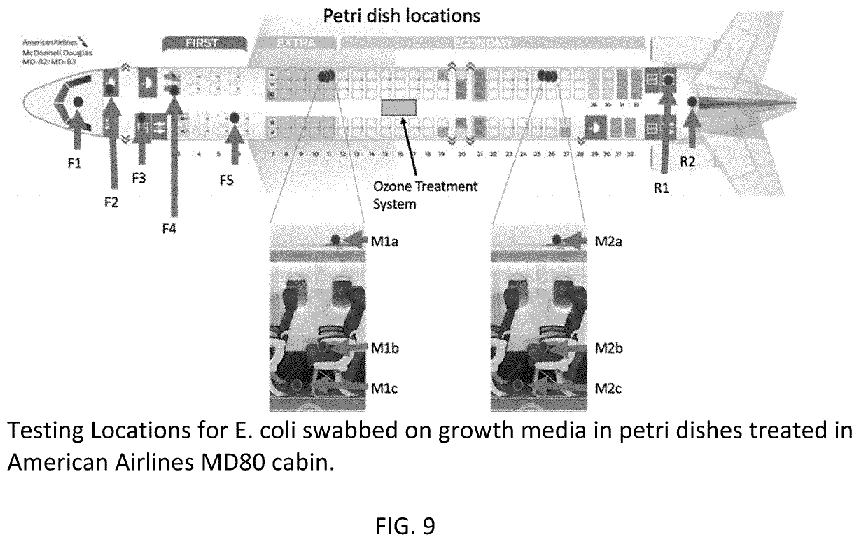

As seen in FIG. 9, the treatment system with ozone generator is placed in the center of the cabin area in the aisle and then allowed to operate as described above. After the test cycle was completed, the effectiveness of the treatment system was determined by analyzing the kill rates of E. coli contaminated petri dishes determined as a percentage in various areas where E. coli were subjected to ozone treatment within the airplane cabin. The test sample locations are shown in FIG. 9. The inventive treatment system was highly effective showing up to 100% inactivation of E. coli samples plated on growth media in the airplane cabin.

In accord with the foregoing disclosure, the inventive treatment system has generated laboratory data showing excellent kill rates and reduced catalyzed decomposition times in the treatment area, such as a room, airplane cabin or ozone chamber. The treatment system is able to generate ozone, kill up to 100% of bacteria, and decompose ozone to 0.1 ppm in approximately 25 minutes or less on small aircraft. In comparison, the treatment system without catalyst can generate ozone, kill over 97% of bacteria, and decompose ozone to 0.1 ppm in approximately 45 minutes on large aircraft. The treatment system is designed to be operated remotely to treat large spaces, which eliminates the requirement for human operation within the treatment space and prevents dangerous exposures to ozone. The treatment system may also be configured as a closable cabinet that can sterilize PPE and other materials.

Although particular preferred embodiments of the invention have been disclosed in detail for illustrative purposes, it will be recognized that variations or modifications of the disclosed apparatus, including the rearrangement of parts, lie within the scope of the present invention.

We hereby claim:

1. A treatment system, which creates and pumps ozone to fill a treatment space containing air, said treatment system comprising:
an ozone generator comprising at least one plate generator connected to an oxygen source for generating ozone, and a passage discharging a flow of said ozone to the treatment space;
a detector comprising a sensor unit which detects and monitors a concentration of said ozone within the treatment space and automatically adjusts operation of said ozone generator and said flow of said ozone into the treatment space to maintain said ozone at a selected concentration which sanitizes the treatment space of pathogens contacted by said ozone.

2. The treatment system according to claim 1, which further comprises an air handling unit that moves air and said ozone to the treatment space to equally distribute ozone during ozone generation.

3. The treatment system according to claim 1, which further comprises a converter unit configured as a catalytic converter to convert said ozone back to oxygen, said converter unit comprising a catalyst to decompose said ozone back to oxygen and reduce said ozone concentration so as to restore the treatment spaces to a condition safe for people faster than would occur through unaided decomposition of said ozone.

4. The treatment system according to claim 3, wherein said catalyst is a Metal-Organic Framework (MOF) serving as a reactive catalyst in said converter unit.

5. The treatment system according to claim 4, wherein said MOF is a substance UiO-66-NHO₂.
